(19) United States
(12) Patent Application Publication
(10) Pub. No.: US 2007/0161785 A1
(43) Pub. Date: Jul. 12, 2007

(54) High density metal ion affinity compositions and methods for making and using the same

(75) Inventors: Grigoriy Simeonov Tchaga, Newark, CA (US); Rajinder K. Bhatia, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: CLONTECH LABORATORIES, INC.

(21) Appl. No.: 11/634,321

(22) Filed: Dec. 4, 2006

Related U.S. Application Data
(60) Provisional application No. 60/742,602, filed on Dec. 5, 2005.

(51) Int. Cl. C07K 1/12

(52) U.S. Cl. 530/414; 525/54.1

(57) ABSTRACT

High density metal ion affinity compositions and methods for making and using the same are provided. The subject compositions include a matrix bonded to ligand/metal ion complexes, where the compositions have a high metal ion density. The subject compositions find use in a variety of different applications. Also provided are kits and systems that include the subject compositions.

### CROSS-REFERENCE To RELATED APPLICATIONS

Pursuant to 35 U.S.C. § 119 (e), this application claims priority to the filing date of U.S. Provisional Patent Application Ser. No. 60/742,602 filed Dec. 5, 2005; the disclosure of which is herein incorporated by reference.

### BACKGROUND

Immobilized Metal Ion Affinity Chromatography (IMAC) is one of the most frequently used techniques for purification of fusion proteins containing affinity sites for metal ions. IMAC is a separation principle that utilizes the differential affinity of proteins for immobilized metal ions to effect their separation. This differential affinity derives from the coordination bonds formed between metal ions and certain amino acid side chains exposed on the surface of the protein molecules.

Since the interaction between the immobilized metal ions and the side chains of amino acids has a readily reversible character, it can be utilized for adsorption and then be disrupted using mild (i.e., non-denaturing) conditions. Adsorbents that are currently commercially available include iminodiacetic acid (IDA), nitriloacetic acid (NTA), caboxymethylated aspartic acid (CM-Asp), and tris-carboxymethyl ethylene diamine (TED). These ligands offer a maximum of tri- (IDA), tetra- (NTA, CM-Asp), and penta-dentate (TED) complexes with the respective metal ion.

### BRIEF DESCRIPTION OF THE FIGURES

FIG. 1: SDS Electrophoresis analyses of the purification of 6×HN N-terminally tagged AcGFP and LacZ with TALON Magnetic beads.

E. coli cells expressing 6×HN-AcGFP or 6×HN-LacZ were extracted in TALON Extractor buffer and mixed with Co²⁺-CM-Asp magnetic beads (TALON Magnetic beads). The beads were equilibrated with 50 mM sodium phosphate, 0.3M NaCl, pH 7.2 followed by wash with 10 mM imidazole in the equilibration buffer. The protein was eluted with 250 mM imidazole in the equilibration buffer.

Panel A: SDS-PAGE analysis of the purification for 6×HN-AcGFP.

Panel B: SDS-PAGE analysis of the purification for 6×HN-LacZ

Lanes are as follows: 1. MW markers, 2. Starting E. coli Extract, 3. Non adsorbed material, 4. Eluted Protein, 5. MW Markers

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The phrase “metal ion affinity composition” refers to a composition of matter having a polymeric matrix bonded to ligand/metal ion complexes, e.g., aspartate-based tetradentate ligand/metal ion complexes, where the metal ion complexes have affinity for proteins, e.g., tagged with a metal ion affinity peptide. In certain embodiments, the affinity composition includes aspartate groups and is referred to as an aspartate-based metal ion affinity composition, where such compositions include a structure that is synthesized from an aspartic acid, e.g., L-aspartic acid. The structure may have four ligands capable of interacting with, i.e., chelating, a metal ion, such that the metal ion is stably but reversibly associated with the ligand, depending upon the environmental conditions of the ligand.

As is known in the art, the compositions may be charged or uncharged. A composition is charged when the ligands thereof are complexed with metal ions. Conversely, a complex is uncharged when the ligands thereof are uncomplexed or free of metal ions, but may be complexed with metal ions.

The phrase “metal ion source” refers to a composition of matter, such as a fluid composition, that includes metal ions. As used herein, the term “metal ion” refers to any metal ion for which the affinity peptide has affinity and that can be used for purification or immobilization of a fusion protein. Such metal ions include, but are not limited to, Ni²⁺, Co²⁺, Fe³⁺, Al³⁺, Zn²⁺ and Cu²⁺. As used herein, the term “hard metal ion” refers to a metal ion that shows a binding preference for oxygen. Hard metal ions include Fe³⁺, Ca²⁺, and Al³⁺. As used herein, the term “soft metal ion” refers to a metal ion that shows a binding preference of sulfur. Soft metal ions include Cu⁺, Hg²⁺, and Ag⁺. As used herein, the term “intermediate metal ion” refers to a metal ion that coordinates nitrogen, oxygen, and sulfur. Intermediate metal ions include Cu²⁺, Ni²⁺, Zn²⁺, and Co²⁺.

As used herein, the term “contacting” means to bring or put together. As such, a first item is contacted with a second item when the two items are brought or put together, e.g., by touching them to each other.

The term “sample” as used herein refers to a fluid composition, where in certain embodiments the fluid composition is an aqueous composition.

As used herein, the phrase “in the presence of” means that an event occurs when an item is present. For example, if two components are mixed in the presence of a third component, all three components are mixed together.

The phrase “oxidation state” is used in its conventional sense, see e.g., Pauling, General Chemistry (Dover Publications, N.Y.) (1988).

The terms “affinity peptide,” “high affinity peptide,” and “metal ion affinity peptide” are used interchangeably herein to refer to peptides that bind to a metal ion, such as a histidine-rich or HAT peptides.

The term “affinity tagged polypeptide” refers to any polypeptide, including proteins, to which an affinity peptide is fused, e.g., for the purpose of purification or immobilization.

As used herein, the terms “adsorbent” or “solid support” refer to a chromatography or immobilization medium used to immobilize a metal ion.

### DETAILED DESCRIPTION

High density metal ion affinity compositions and methods for making and using the same are provided. The subject compositions include a matrix bonded to ligand/metal ion complexes, where the compositions have a high metal ion density. The subject compositions find use in a variety of different applications. Also provided are kits and systems that include the subject compositions.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, certain illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the invention include high density metal ion affinity compositions, as well as methods for their preparation and use. In further describing the subject invention, the subject compositions and their preparation are described first in greater detail, followed by a review of illustrative applications in which they find use. Also provided is a review of the kits and systems.

High Density Metal Ion Affinity Compositions

As summarized above, the present invention provides high density metal ion affinity compositions. The subject compositions are characterized by having a polymeric matrix (i.e., substrate) bonded to ligand/metal ion complexes, e.g., aspartate-based tetradentate ligand/metal ion complexes. By “aspartate-based tetradentate ligand” is meant a structure that is synthesized from an aspartic acid, e.g., L-aspartic acid, where the structure has four ligands capable of interacting with a metal ion. As such, by “tetradentate ligand” is meant that the ligand chelates a metal ion by occupying up to four, and typically four, coordination sites of a metal ion. For example, where a given metal ion has six coordination sites, four of them can be occupied simultaneously by the ligands of the subject tetradentate ligands.

In certain embodiments, the aspartate-based tetradentate ligand of the subject compositions is an alkylaspartate ligand, generally a lower alkylaspartate ligand, such as a 1 to 6, e.g., a 1 to 4, carbon atom alkylaspartate ligand, where the alkyl moiety may or may not be substituted. Representative alkylaspartate ligands of interest include, but are not limited to: carboxymethylated aspartate ligand, carboxyethylated aspartate ligand, etc.

As summarized above, the aspartate-based tetradentate ligand of the subject metal ion high affinity compositions is bonded to, either directly or through a linking group (also referred to herein as a spacer), a matrix (i.e., a substrate or carrier). Matrices of interest include, but are not limited to, polymeric matrices, such as cross-linked polymeric matrices, e.g., dextrans, polystyrenes, nylons, agaroses, and polyacrylamides. Non-limiting examples of suitable, commercially available matrices include, but are not limited to: Sepharose®6B-CL (6% cross-linked agarose; Pharmacia); Superflow™ (6% cross-linked agarose; Sterogene Bioseparations, Inc.), Uniflow™ (4% cross-linked agarose; Sterogene Bioseparations, Inc.); silica matrices; magnetic beads, e.g., agarose magnetic beads; and the like.

In certain embodiments, the matrix component is bonded, optionally through a linking group, to the above-summarized aspartate-based tetradentate ligand/metal ion complexes. In certain embodiments, the tetradentate ligands may be bonded, such as covalently bonded, to the matrix either directly or through a linking group. Where linking groups are employed, such groups are chosen to provide for covalent attachment of the ligand to the matrix through the linking group. Linking groups of interest may vary widely depending on the nature of the matrix and ligand moieties. The linking group, when present, may be biologically inert. In certain embodiments, the size of the linker group, when present, is generally at least about 50 daltons, such as at least about 100 daltons and included at least about 1000 daltons or larger, an in certain embodiments does not exceed about 500 daltons and in certain embodiments does not exceed about 300 daltons. Generally, such linkers include a spacer group terminated at either end with a reactive functionality capable of covalently bonding to the substrate or ligand moieties. Spacer groups of interest include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject molecules include heterofunctional compounds, such as azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

The aspartate-based tetradentate ligands are in certain embodiments bonded to the matrices at a ratio of tetradendate ligand to residue, e.g., glucose unit, that provides for acceptable characteristics, where the ratio of tetradentate ligand to polymeric matrix residue may range from about 1 tetradentate ligand for every about 1 to 100 residues, e.g., from about 1 tetradentate ligand for every about 5 to 50 residues, including 1 tetradentate ligand for every about 10 to about 20 residues.

As reviewed above, in charged versions of the affinity compositions, the ligands, e.g., aspartate-based tetradentate ligands, are complexed with metal ions. In other words, the tetradenate ligands are “charged with” metal ions. Said yet another way, metal ions are chelated by the tetradentate ligands of the compositions.

A variety of different types of metal ions may be complexed to the ligands of the subject compositions. A variety of different types of metal ions may be complexed to the ligands of the subject compounds. Metal ions of interest can be divided into different categories (e.g., hard, intermediate and soft) based on their preferential reactivity towards nucleophiles. Hard metal ions of interest include, but are not limited to: Fe³⁺, Ca²⁺ and Al³⁺ and like. Soft metal ions of interest include, but are not limited to: Cu⁺, Hg²⁺, Ag⁺, and the like. Intermediate metal ions of interest include, but are not limited to: Cu²⁺, Ni²⁺, Zn²⁺, Co²⁺ and the like. In certain embodiments, the metal ion that is chelated by the ligand is Co²⁺. In certain embodiments, the metal ion of interest that is chelated by the ligand is Fe³⁺. Additional metal ions of interest include, but are not limited to lanthanides, such as Eu³⁺, La³⁺, Tb³⁺, Yb³⁺, and the like.

A feature of certain embodiments of the subject invention is that compositions are high density metal ion affinity compositions. By “high density) is meant that the density of the metal ions of the composition is greater, e.g., by at least about 10%, such as by at least about 20%, including by at least about 50% or more, such as by at least about 100% or more, than the density that is present on compositions produced according to other fabrication protocols in which the matrix is activated with a non-divinyl sulfone activator, e.g., where the matrix is activated via epoxy activation as described in U.S. Pat. Nos. 6,242,581 and 5,962,641. In representative embodiments, the metal ion density of the affinity compositions is at least about 25 μmol/ml of swollen affinity composition, such as at least about 30 μmol/ml swollen affinity composition, including at least about 35 μmol/ml swollen affinity composition, e.g., 39 μmol or greater/ml swollen affinity composition, as determined using the density determination protocol described in the Experimental Section, below.

In certain embodiments, the water-soluble metal ion affinity composition has the following structure: 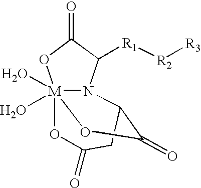
wherein:

M is a metal ion;

R₁=a linking arm connecting the methylene carbon atom of the carboxymethyl group of the CM-Asp moiety with R₂;

R₂=linker that links R1 to R₃; and

R₃=a polymeric matrix.

Of particular interest in certain embodiments are the metal ion chelating compositions disclosed in U.S. Pat. Nos. 6,703,498; 6,242,581 and 5,962,641, as well as U.S. patent application Ser. No. 09/920,684 published as US 2002/0019496; and U.S. patent application Ser. No. 11/249,151; the disclosures of which are herein incorporated by reference, where the compositions described in these patents and applications are ones modified as described herein to be high density metal ion affinity compositions.

The subject compositions can be provided in the form of a chromatography column, e.g., wherein the composition is packed in a column. The composition can also comprise a structure that is a solid support of any shape or configuration. Thus, the composition can be in any form, e.g., a bead, a sheet, a well, and the like. The term bead is meant broadly to include any small structure, where the structure may be spherical or non-spherical, including egg shaped, flattened spherical, or irregular shaped. Where the composition is a bead, the beads are provided in various sizes, depending, in part, on the nature of the sample being applied, where suitable bead sizes include those having a longest dimension, e.g., diameter, from about 10 μm to about 500 μm, e.g., from about 10 μm to about 20 μm, from about 16 μm to about 24 μm, from about 20 μm to about 50 μm, from about 50 μm to about 100 μm, from about 60 μm to about 160 μm, from about 100 μm to about 200 μm, from about 100 μm to about 300 μm, from about 200 μm to about 300 μm, or from about 300 μm to about 500 μm. In certain embodiments, the solid support, e.g., bead, may be a magnetic bead. Non-limiting examples of formats in which a composition is provided include a gravity-flow column; a fast protein liquid chromatographic (FPLC) column; a multi-well (e.g., 96-well) column format; a spin column; and the like.

Methods of Fabrication

Aspects of the invention include preparing high density metal ion affinity compositions. In certain embodiments, the methods employ divinyl sulfone activation. In these embodiments, a polymeric matrix is first contacted with a divinyl sulfone (DVS) activating composition under conditions sufficient to provide an activated polymeric matrix. Matrices of interest include, but are not limited to, polymeric matrices, such as cross-linked polymeric matrices, e.g., including polysaccharides, e.g., dextrans, agaroses, etc., as well as other polymeric matrices, e.g., and polystyrenes, nylons, polyacrylamides. Non-limiting examples of suitable, commercially available matrices include, but are not limited to: Sepharose®6B-CL (6% cross-linked agarose; Pharmacia); Superflow™ (6% cross-linked agarose; Sterogene Bioseparations, Inc.), Uniflow™ (4% cross-linked agarose; Sterogene Bioseparations, Inc.); silica matrices; magnetic beads, e.g., agarose magnetic beads; and the like.

In certain embodiments, the divinyl sulfone activating composition is contacted with the matrix in a ratio ranging from about 1 to about 20 ml DVS composition/100 grams matrix, such as from about 2 to about 10 ml DVS composition/100 grams matrix, including from about 5 to about 10 ml DVS composition/100 grams matrix. The DVS composition that is contacted with the matrix may be any convenient DVS composition, where the composition is, in certain embodiments, a fluid composition, such as an aqueous fluid composition, where the concentration of DVS in the fluid composition may range from about 1% to about 20% such as from about 2% to about 10%, including from about 5% to about 10%. The DVS composition has, in certain embodiments, a pH ranging from about 9 to about 13, such as from about 11 to about 12. Contact between the matrix and the DVS activating composition is maintained for a period of time sufficient for the desired amount of activation to occur, e.g., from about 0.5 hr to about 4 hrs, such as from about 1 hr to about 2 hrs, where contact is maintained a suitable temperature, e.g., from about 4° C. to about 40° C., such as from about 25° C. to about 30° C., e.g., room temperature. In certain embodiments, the activating composition and matrix are contacted with agitation, e.g., stirring. Contact of the DVS activating composition and matrix results in the production of an activated polymeric matrix.

The resultant activated matrix is then contacted with an aspartic acid composition, e.g., a fluid comprising L-aspartic acid, to produce an aspartate-polymeric matrix conjugate. In certain embodiments, the aspartic acid composition is contacted with the matrix in a ratio ranging from about 50 to about 1000 ml aspartic acid composition/100 grams activated matrix, such as from about 100 to about 300 ml aspartic acid composition/grams activated matrix, including from about 100 to about 200 ml aspartic acid composition/100 grams activated matrix. The aspartic acid composition that is contacted with the matrix may be any convenient aspartic acid composition, where the composition is, in certain embodiments, a fluid composition, such as an aqueous fluid composition, where the concentration of aspartic acid in the fluid composition may range from about 0.1M to about 1.0M, such as from about 0.5M to about 1.0M, including from about 0.8M to about 1.0M. The aspartic acid composition has, in certain embodiments, a pH ranging from about 9 to about 13, such as from about 10 to about 11. Contact between the matrix and the aspartic acid composition is maintained for a period of time sufficient for the desired amount of activation to occur, e.g., from about 12 hrs to about 48 hrs, such as from about 12 hrs to about 16 hrs, where contact is maintained a suitable temperature, e.g., from about 4° C. to about 40° C., such as from about 25° C. to about 30° C., e.g., room temperature. In certain embodiments, the aspartic acid composition and matrix are contacted with agitation, e.g., stirring. Contact of the aspartic acid composition and matrix results in the production of an aspartate-polymeric matrix conjugate.

Aspects of the invention include contacting the resultant aspartate-polymeric matrix conjugate with an alkylating composition that includes an alkylating agent to produce an alkylated-aspartate polymeric matrix, which is also referred to herein as an uncharged affinity composition. In certain embodiments, the alkylating agent is one that reacts with the aspartate moiety of the aspartate-polymeric matrix to produce an alkylaspartate ligand, generally a lower alkylaspartate ligand, such as a 1 to 6, e.g., a 1 to 4, carbon atom alkylaspartate ligand, e.g., carboxymethylated aspartate ligand, carboxyethylated aspartate ligand, etc., where the alkyl moiety may or may not be substituted. Representative alkylating agents of interest include, but are not limited to: bromoacetic acid, bromopropionic acid and the like.

In certain embodiments, the alkylating composition is contacted with the aspartate-polymeric matrix conjugate in a ratio ranging from about 100 to about 1000 ml alkylating composition/grams matrix conjugate, such as from about 100 to about 300 ml alkylating composition/grams matrix conjugate, including from about 100 to about 200 ml alkylating composition/grams matrix conjugate. The alkylating composition that is contacted with the matrix-conjugate may be any convenient alkylating composition, where the composition is, in certain embodiments, a fluid composition, such as an aqueous fluid composition, where the concentration of alkylating agent in the fluid composition may range from about 0.5M to about 2.0M, such as from about 1.0M to about 1.8M, including from about 1.5M to about 1.8M. The alkylating composition has, in certain embodiments, a pH ranging from about 9 to about 14, such as from about 10 to about 11. Contact between the matrix-conjugate and the alkylating composition is maintained for a period of time sufficient for the desired amount of alkylation to occur, e.g., from about 24 hrs to about 72 hrs, such as from about 43 hrs to about 60 hrs, where contact is maintained a suitable temperature, e.g., from about 4° C. to about 40° C., such as from about 25° C. to about 30° C., e.g., room temperature. In certain embodiments, the alkylating composition and matrix-conjugate are contacted with agitation, e.g., stirring. Contact of the alkylating composition and matrix-conjugate results in the production of an uncharged affinity composition, e.g., one that includes tetradentate ligands.

Aspects of the invention also include charging the uncharged affinity composition with a metal ion. In these embodiments, an uncharged composition, e.g., as described above, is contacted with a source of metal ions in a manner such that metal ions are complexed by the ligands of the uncharged composition to produce a charged composition. To charge the uncharged composition with metal ion, the uncharged composition is contacted with a source of metal ions.

In certain embodiments, the source of metal ions is an aqueous fluid composition that includes acetic acid. The concentration of metal ion in the fluid, e.g., aqueous, composition may vary, but ranges from about 2 mM to about 250 mM, such as from about 10 mM to about 50 mM, including from about 20 mM to about 50 mM, in certain embodiments. In certain embodiments, the metal ion is a hard, intermediate and soft metal ion. Hard metal ions of interest include, but are not limited to: Fe³⁺, Ca²⁺ and Al³⁺ and like. Soft metal ions of interest include, but are not limited to: Cu⁺, Hg²⁺, Ag⁺, and the like. Intermediate metal ions of interest include, but are not limited to: Cu²⁺, Ni²⁺, Zn²⁺, Co²⁺ and the like. In certain embodiments, the metal ion that is chelated by the ligand is Co²⁺. In certain embodiments, the metal ion of interest that is chelated by the ligand is Fe³⁺. Additional metal ions of interest include, but are not limited to lanthanides, such as Eu³⁺, La³⁺, Tb³⁺, Yb³⁺, and the like. The metal ion source has, in certain embodiments, a pH ranging from about 2.0 to about 7.0, such as from about 2.0 to about 3.0. The resultant mixture is maintained at a sufficient temperature, e.g., from about 4° C. to about 40° C., such as from about 15° C. to about 25° C., for a sufficient period of time, e.g., from about 5 min to about 48 hrs such as from about 20 min to about 60 min, to produce the desired charged composition. Where desired, the reaction mixture may be agitated, e.g., via mixing.

The resultant charged composition is then washed to remove excess metal ion. Any convenient washing protocol may be employed. Where desired, e.g., where the composition is to be stored for a period of time prior to use, the charged composition may be stabilized and placed into a storage medium. Any convenient stabilization protocol may be employed, such as the protocol disclosed in U.S. application Ser. No. 11/249,151; the disclosure of which is herein incorporated by reference.

Where desired, the resultant stabilized composition is combined with a storage medium. Any convenient storage medium may be employed. In certain embodiments, the storage medium is an aqueous solution of a lower alcohol, e.g., ethanol. In representative embodiments, the storage medium is a fluid that ranges from about 10 to about 90% alcohol, such as from about 15 to about 75% alcohol, including from about 20 to about 50% alcohol, e.g., 25% alcohol.

Utility

The subject metal ion affinity compositions find use in a number of different applications. Such applications include, but are not limited to, purification applications. As such, one type of application in which the subject metal ion affinity compositions find use is purification. Specifically, the subject metal ion affinity compounds find use in the purification of analytes that have an affinity for a chelated metal ions, e.g., chelated metal ions in a 2+ oxidation state with a coordination number of 6. The term purification is used broadly to refer to any application in which the analyte (i.e., target molecule) is separated from its initial environment, e.g., sample in which it is present, and more specifically the other components of its initial environment. In embodiments of the purification applications, the protocol employed includes: contacting a fluid sample that includes the analyte of interest with the metal ion affinity composition under conditions sufficient for any analytes having affinity for the chelated metal ion to bind to the metal ion component of the metal ion affinity composition. In other words, the metal ion affinity composition and sample are combined under conditions sufficient to produce complexes between the analyte and the water-soluble compound in a resultant mixture. As reviewed above, the metal ion affinity composition may be part of insoluble support, e.g., a bead, plate, well of a microtitre plate, etc, as described above. Alternatively, the metal ion affinity composition may be free in solution, e.g., where it has been solubilized according to the solubilization protocol disclosed in U.S. Pat. No. 6,703,498; the disclosure of which is herein incorporated by reference.

Following this initial step, any resultant complexes are separated from the remainder of the initial sample. Separation may be achieved in a number of different ways, including two-phase separation protocols, separation based on weight, magnetic properties, e.g., centrifugation protocols, electrophoretic protocols, etc; chromatographic protocols, etc.

Analytes that may be purified according to the subject methods include metal ion affinity peptide tagged compounds. In certain embodiments, the analytes of interest include a metal ion affinity tag, e.g., they are fusion proteins having a metal ion affinity tag domain, where particular metal ion affinity tags of interest include tags that have one or more histidine residues, e.g., poly-his containing affinity peptides. Representative metal ion affinity peptides of interest include those described in U.S. Pat. No. 4,569,794 and U.S. Pat. No. 5,594,115, as well as pending U.S. patent application Ser. No. 09/858,332; the disclosures of which are herein incorporated by reference.

In certain embodiments, the affinity peptide portion is a histidine-rich polypeptide sequence with a general sequence: (XHYZ)ₙ, wherein X and Y=any amino acid except histidine, Z=any amino acid, and n=2 or more. In yet other embodiments, the affinity peptide comprises a peptide of the formula (His-X₁-X₂)ₙ₁-(His-X₃-X₄-X₅)ₙ₂-(His-X₆)ₙ₃, wherein each of X₁ and X₂ is independently an amino acid with an aliphatic or an amide side chain, each of X₃, X₄, X₅ is independently an amino acid with a basic or an acidic side chain, each X₆ is an amino acid with an aliphatic or an amide side chain, n1 and n2 are each independently 1-3, and n3 is 1-5. In some embodiments, the affinity peptide has the amino acid sequence NH₂-His-Leu-Ile-His-Asn-Val-His-Lys-Glu-Glu-His-Ala-His-Ala-His-Asn-COOH (i.e., a HAT sequence). In certain embodiments, the affinity peptide has the formula (His-Asn)ₙ, where n=3 to 10. In one particular embodiment, n=6. In certain embodiments, the affinity peptide has the formula (His-X₁-X₂)ₙ, wherein each of X₁ and X₂ is an amino acid having an acidic side chain, and n=3 to 10. In one embodiment, the affinity peptide comprises the sequence (His-Asp-Asp)₆. In another embodiment, the affinity peptide comprises the sequence (His-Glu-Glu)₆. In a further embodiment, the affinity peptide comprises the sequence (His-Asp-Glu)₆. These affinity peptides and methods for making analytes, e.g., fusion proteins, tagged with the same are further described in U.S. patent application Ser. No. 09/858,332, filed on May 15, 2001 and titled “Metal Ion Affinity Tags And Methods Of Use Thereof”; the disclosure of which is herein incorporated by reference.

In certain embodiments, following separation of the complexes from the remainder of the initial sample, the analyte is separated from the metal ion affinity component. The analyte may be separated from the metal ion affinity component using any convenient protocol, where suitable protocols include changing the conditions, e.g., salt concentration etc, of the environment to achieve dissociation of the analyte from the chelated metal ion.

In certain embodiments, the subject water-soluble metal ion affinity complexes are present as a solid support and employed as solid support bound affinity reagents for purifying one or more analytes from a sample. In such embodiments, the solid supports are contacted with the sample so that any analytes having affinity for the metal ion affinity compounds bind to the metal ion/ligand complexes of the solid support. The resultant solid support bound complexes are then separated from the remainder of the mixture to obtain purified analyte, which can then be further separated from the solid support immobilized water soluble metal ion affinity compounds, as described above.

In addition to the above-described representative applications, the affinity compositions may also find use in IMAC affinity peptide tagged protein purification protocols, such as those described in U.S. Pat. Nos. 4,569,794; 5,047,513; 5,284,933; 5,310,663; 5,962,641; 5,594,115; and 6,242,581; the disclosures of which are herein incorporated by reference, as well as the purification and analyte detection applications described in U.S. Pat. No. 6,703,498 and the phosphoprotein enrichment protocols, as described U.S. patent application Ser. No. 11/249,151; the disclosures of which protocols are herein incorporated by reference.

Kits and Systems

Aspects of the invention also include kits and systems for use in practicing the subject methods. The kits and systems at least include the metal ion affinity compositions, as described above. The kits and systems may also include a number of optional components that find use in the subject methods. Optional components of interest include buffers, including extraction/loading/washing buffer or buffers (e.g., as described above), and the like. Furthermore, the kits and systems may include reagents for producing affinity peptide tagged polypeptides, e.g., vectors encoding metal ion affinity peptides, such as those disclosed in U.S. patent application Ser. No. 09/858,332; the disclosure of which vectors are incorporated herein by reference.

In certain embodiments, the kits will further include instructions for practicing the subject methods or means for obtaining the same (e.g., a website URL directing the user to a webpage which provides the instructions), where these instructions are typically printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation.

### Experimental

I. Protocol for Preparation of TALON Magnetic Beads

A. Twenty mL of magnetic 4% agarose beads are washed with Milli Q water to remove storage buffer. The separation of the liquid and solid phases (washing solution and magnetic beads) is achieved by placing of the flask on a magnetic separator. After the beads have settled down, the supernatant is aspirated off while keeping the flask on the magnetic separator.

B. DVS Activation of Magnetic Agarose Beads

The magnetic beads are transferred to a fresh 250 mL conical flask with 20 mL of 1.0 M Na₂CO₃. The flask is placed on a magnetic separator. After the beads have settled down, the supernatant is aspirated off while keeping the flask on the magnetic separator. Twenty mL of 1.0 M Na₂CO₃ and 1.0 mL of Divinyl sulfone are added. The mixture is left on an orbital shaker at RT. After 2 hours the flask is removed from the orbital shaker and placed on a magnetic separator. After the beads have settled down, the supernatant is aspirated off while keeping the flask on the magnetic separator.

C. Coupling of Aspartic Acid

The DVS-activated magnetic beads are washed extensively with Milli Q water using a magnetic separator until the pH of the supernatant is same as pH of water. Sodium hydroxide (NaOH)-0.85 g is dissolved in 20 mL Milli Q water with mechanical stirring in a 250 ml flask. The NaOH solution can be stored in a refrigerator and before starting the coupling of aspartic acid is placed in an ice bath. 1.8 g L-aspartic acid (MW 133.1) is added in with stirring, followed by 5.3 g sodium carbonate (MW 106) with stirring. The temperature is monitored and if it is higher than 25° C., the solution is cooled to 25° C. in an ice bath. The pH of the solution is adjusted to the range 11.0-11.1 by the addition of 10N NaOH or 6N HCl. The washed beads are transferred to a 250 ml flask using 10% Na₂CO₃. Remove the Na₂CO₃ from the beads using a magnetic separator. The aspartic acid solution is transferred to the flask containing the magnetic beads and the reaction is carried out on an orbital shaker at ambient temperature for 16 hours. The beads are washed with Milli Q water until the pH of washes is same as pH of water.

D. Carboxy-Methylation

1.65 g NaOH is dissolved in 22 mL Milli Q water with mechanical stirring in a 250 ml flask. The NaOH solution can be stored in a refrigerator and before starting the coupling of aspartic acid is placed in an ice bath. 5.5 g bromoacetic acid (MW 139) is added in 1 g increments, with stirring. The temperature of solution during the addition is monitored; the temperature should be no higher than 30° C. at the end of the addition. Before proceeding further, the pH of the solution is measured and if the pH is lower than 7, it is adjusted by adding NaOH pellets, 0.5 g at a time, being careful not to let the temperature exceed 30° C., until the solution pH is equal or higher than 7. Carefully 1.2 g Na₂CO₃ (MW 106) is added with stirring, and the flask containing the solution is removed from the ice bath; the Na₂CO₃ goes completely into solution as the solution warms. The pH is adjusted to the range of 10.0-10.1 with conc. HCl or conc. NaOH, using a calibrated pH meter. The magnetic beads are transferred using 10% Na₂CO₃ to a conical 250 mL flask. The bromoacetic acid solution is added to the flask and the suspension is mixed at ambient temperature for at least 43 hours. The flask is placed on a magnetic separator. After the beads have settled down, the supernatant is aspirated off while keeping the flask on the magnetic separator. The beads are washed thoroughly with 4×100 mL Milli Q water, 1×50 mL 10% acetic acid, and finally with Milli Q water until the pH of washes is same as the pH of water. The beads can then either be charged with metal ion immediately or stored in 25% ethanol.

II. Charging of the CM-Asp Magnetic Beads with Metal Ion

20 mL of the final beads produced in Example I, above, are charged with 100 mL of freshly prepared 50 mM CoCl₂.6H₂O in Milli Q water for 12 hrs. Beads are washed multiple times with Milli Q water and then stored as 5% suspension in 25% Ethanol

Metal Ion Analysis

2 mL of 5% suspension of TALON Magnetic beads is placed in a pre-weighed tube. The tube is placed on a magnetic separator and storage buffer is removed. The magnetic beads are washed with Milli Q water. The tube with beads is weighed after removal of Milli Q water. The weight of the beads is determined by subtracting the weight of the empty tube from the weight of tube with the beads.

4 mL of 500 mM EDTA, pH 8 is added to the tube and mixed on a rotary mixer overnight at RT. The sample is centrifuged for 5 minutes at 1000×g and 4 mL of the supernatant is collected. The sample is analyzed for Cobalt after acid digestion using Atomic absorption spectrophotometer.

TALON magnetic beads synthesized according to the protocol in section I and II contain approximately 25 μmol of Cobalt/per 1 g of beads

III. Use of TALON Magnetic Beads for Purification of Polyhistidine Tagged Protein

A) Protocol for Running Samples on TALON Magnetic Beads

Extractor Buffer:

50 mM sodium phosphate (Na₂HPO₄.7H₂O), 300 mM NaCl, 1% non ionic detergents, pH 7.2

Equilibration Buffer:

50 mM sodium phosphate (Na₂HPO₄.7H₂O), 300 mM NaCl, pH 7.0

Wash Buffer:

50 mM sodium phosphate (Na₂HPO₄.7H₂O), 300 mM NaCl, 10 mM imidazole, pH 7.0

Elution Buffer:

50 mM sodium phosphate (Na₂HPO₄.7H₂O), 300 mM NaCl, 250 mM imidazole, pH 7.0

Proteins are extracted from cells by re-suspending the cell pellet in the TALON Extractor buffer and incubating the suspension at 4° C. for 10 min. Cell extract is centrifuged at 10,000×g for 20 min at 4° C. to pellet any insoluble material. The supernatant is transferred to a clean tube.

10 mg of TALON Magnetic beads are used for each experiment. 200 μL of a 5% suspension of TALON magnetic beads is placed in a 1.5 mL tube. The tube is placed on a magnetic separator for one minute. The buffer is aspirated. The magnetic beads are washed with Milli Q water to remove residual storage buffer using the magnetic separator. The beads are equilibrated with 0.5 mL of Equilibration buffer. The clarified cell extract collected above is added to the beads (a small portion of the cell lysate is retained for protein assay and other analysis). The beads with sample are mixed at RT for 30 min on a Rotary shaker. If the target protein is susceptive to proteolysis, the beads are mixed with the sample at 4° C. for 1 hr.

The beads are then placed on a magnetic separator and the non adsorbed extract is collected. The magnetic beads are washed twice with 0.5 mL of equilibration buffer and one wash with 10 mM imidazole in the equilibration buffer to remove any non-adsorbed proteins. Histidine tagged protein is eluted with elution buffer.

B) Material Balance of the Fractions Obtained During the Purification of 6×HN-Tagged AcGFP and LacZ Using Talon Magnetic Beads

The polyhistidine-tagged proteins were expressed in BL21 E. coli cells and extracted in the TALON Extractor buffer. The proteins were run on TALON Magnetic Beads according to the protocol given above (III A)

| | Sample loaded | | Flow-through | | Eluate | |
|---|---|---|---|---|---|---|
| | Protein | Fluorescence¹ | Protein | Fluorescence¹ | Protein | Fluorescence¹ |
| Protein | (mg) | (RFU) | (mg) | (RFU) | (mg) | (RFU) |
| 6xHN-AcGFP | 1.34 | 15,425 | 1.18 | 3.015 | 0.15 | 10,750 |
| 6xHN-LacZ | 1.23 | | 1.16 | | 0.05 | |

¹Relative Fluorescence Units (RFU) for 6xHN-AcGFP

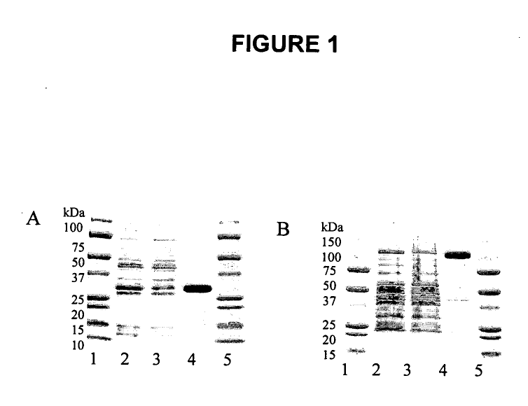

Pierce BCA protein assay (cat#23235) and Bradford protein assay from Bio-Rad (cat#500-0006) was used for protein quantitation.
C) SDS-Electrophoresis Analyses of the Fractions Obtained During the Purification of 6×HN-Tagged AcGFP and LacZ Using TALON Magnetic Beads is Shown in FIG. 1.
High Density Metal Ion Agarose Based Resins
TALON Superflow and Sepharose 6B-CL resins activated with Diviny sulfone based chemistry were synthesized according to the above mentioned protocol in section I.
The Resins were charged either with CoCl₂.6H₂O or with ZnCl₂ according to protocol in Section II. Samples were then analyzed for metal content.

Metal Ion Analysis Results

| | | Amount of Metal/ml of |
|---|---|---|
| Resin | Chemistry | swollen Resin |
| TALON Superflow | DVS | 39 μmol of Co |
| TALON Superflow | Epoxy* | 16 μmol of Co |
| TALON Superflow | DVS | 47 μmol of Zn |

*Reference: U.S. Pat. No. 5,962,641

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of making a metal ion affinity composition, said method comprising:
(a) contacting a polymeric matrix with divinyl sulfone to produce an activated polymeric matrix;
(b) contacting said activated polymeric matrix with aspartic acid to produce an aspartate-polymeric matrix conjugate;
(c) contacting said aspartate-polymeric matrix conjugate with an alkylating agent to produce an uncharged affinity composition; and
(d) contacting said uncharged affinity composition with a metal ion source to produce said metal ion affinity composition.

2. The method according to claim 1, wherein said polymeric matrix is a polysaccharide.

3. The method according to claim 2, wherein said polysaccharide is agarose.

4. The method according to claim 1, wherein said metal ion source comprises a hard metal ion.

5. The method according to claim 4, wherein said hard metal ion is one of Fe³⁺, Ca²⁺ and Al³⁺.

6. The method according to claim 1, wherein said metal ion source comprises an intermediate metal ion.

7. The method according to claim 6, wherein said intermediate metal ion is one of Co²⁺, Ni²⁺, Cu²⁺, or Zn²⁺.

8. The method according to claim 1, wherein said metal ion source comprises a soft metal ion.

9. The method according to claim 8, wherein said soft metal ion is one of Cu⁺, Hg²⁺ and Ag⁺.

10. The method according to claim 1, wherein said metal ion source comprises a lanthanide ion.

11. The method according to claim 4, wherein said lanthanide ion is Eu³⁺.

12. The method according to claim 1, wherein said metal ion source comprises Co²⁺.

13. The method according to claim 1, wherein said alkylating agent comprises bromoacetic acid.

14. The method according to claim 13, wherein said uncharged affinity composition comprises tetradentate ligands.

15. The method according to claim 1, wherein said metal ion affinity composition has the formula: 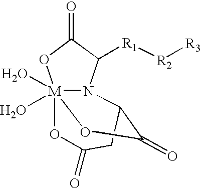
wherein:
M is a transition metal ion in a 2+ oxidation state with a coordination number of 6;
R₁ is a linking arm connecting a methylene carbon atom of a carboxymethyl group with R₂;
R₂ is a linking group linking R1 to R₃; and
R₃=a polymeric matrix.

16. An uncharged affinity composition produced by a method comprising:
(a) contacting a polymeric matrix with divinyl sulfone to produce an activated polymeric matrix;
(b) contacting said activated polymeric matrix with aspartic acid to produce an aspartate-polymeric matrix conjugate;
(c) contacting said aspartate-polymeric matrix conjugate with an alkylating agent to produce said uncharged affinity composition.

17. The uncharged affinity composition according to claim 16, wherein said alkylating agent comprises bromoacetic acid.

18. A high density metal ion affinity composition produced according to the method of claim 1.

19. The high density metal ion affinity composition according to claim 18, wherein said composition has a metal ion density of at least about 35 μmol/ml of swollen composition

20. The high density metal ion affinity composition according to claim 19, wherein said composition comprises Co²⁺.

21. The high density metal ion affinity composition according to claim 20, wherein said metal ion affinity composition is an insoluble structure.

22. The high density metal ion affinity composition according to claim 21, wherein said insoluble structure is a bead.

23. The high density metal ion affinity composition according to claim 22, wherein said bead is a magnetic bead.

24. A method of separating an analyte having affinity for a chelated metal ion from other components of a sample, said method comprising:
(a) contacting said sample with a high density metal ion affinity composition according to claim 1 to produce a contacted mixture; and
(b) separating complexes between said analyte and said high density metal ion affinity composition from other components in said contacted mixture to separate said analyte from other components of said sample.

25. The method according to claim 24, wherein said analyte is tagged with a metal ion affinity peptide.

26. The method according to claim 25, wherein said metal ion affinity peptide is chosen from a multiple histidine residue peptide and a HAT peptide.

27. The method according to claim 26, wherein said method further comprises separating said analyte from said high density metal ion affinity composition.

28. A kit comprising:
a high density metal ion affinity composition according to claim 18; and
a vector encoding a metal ion affinity peptide.
